(19) United States
(12) Patent Application Publication
(10) Pub. No.: US 2023/0405051 A1
(43) Pub. Date: Dec. 21, 2023

(54) PHARMACEUTICAL COMPOSITION FOR PREVENTION OR TREATMENT OF RHEUMATOID ARTHRITIS, COMPRISING, AS ACTIVE INGREDIENT, STEM CELLS HAVING EXPRESSION OF SPECIFIC GENES INCREASED OR DECREASED THEREIN

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Seung Ki KWOK, Seoul (KR); Jaeseon LEE, Seoul (KR); Se Gwang JANG, Seoul (KR); Jung Yeon LIM, Seoul (KR); Sun Hwa PARK, Seoul (KR); Jung Ho CHUN, Seoul (KR)

(21) Appl. No.: 18/250,087

(22) PCT Filed: Oct. 22, 2021
(86) PCT No.: PCT/KR2021/014926

§ 371 Date: Apr. 21, 2023

(30) Foreign Application Priority Data
Oct. 22, 2020 (KR) 10-2020-0137722
Oct. 21, 2021 (KR) 10-2021-0141395

(51) Int. Cl. A61K 35/28; A61P 19/02

(52) U.S. Cl. CPC A61K 35/28; A61P 19/02

(57) ABSTRACT

The present invention relates to a pharmaceutical composition for prevention or treatment of rheumatoid arthritis, the composition comprising, as an active ingredient, human nasal inferior turbinate-derived stem cells in which the expression of specific genes is increased or decreased. The human nasal inferior turbinate-derived stem cells exhibiting a therapeutic effect on arthritis were identified to specifically have an increased expression level of HAS2, CXCL1, or KRTAP1-5 gene or a decreased expression level of GSTT2B or C4B gene. Thus, by taking advantage of this feature, only stem cells that have a therapeutic effect on arthritis can be selected for use in treating rheumatoid arthritis, with the expectation of enhancing the therapeutic effect. Furthermore, it is expected that a method for selecting stem cells useful as a therapeutic agent for rheumatoid arthritis can be provided.

### TECHNICAL FIELD

The present invention relates to a pharmaceutical composition for preventing or treating rheumatoid arthritis, which comprises stem cells in which the expression of a specific gene is decreased or increased as an active ingredient.

This application claims priority to and the benefit of Korean Patent Application No. 10-2020-0137722, filed on Oct. 22, 2020, and Korean Patent Application No. 10-2021-0141395, filed on Oct. 21, 2021, the disclosures of which are incorporated herein by reference in their entirety.

### BACKGROUND ART

Stem cells refer to cells that have “pluripotent” which is the ability to theoretically differentiate into all types of functional cells, “a self-renewal property” to generate cells that have the same morphology and capability as themselves, and a “homing effect” in which stem cells find a damaged site when administered in vivo. Stem cells may be largely classified into adult stem cells and embryonic stem cells, and unlike embryonic stem cells, adult stem cells do not have ethical restrictions and there is no possibility of developing tumors such as teratomas, so research on adult stem cells as cell therapeutic agents is being actively conducted.

Nasal inferior turbinate tissues are independent small bones showing shell-like structures at the lower lateral sides of the left and right nasal cavities and attached to the maxilla and the palatine bone. The inventors recently reported that mesenchymal stem cells, which are the most representative adult stem cells, isolated from disposed human nasal inferior turbinate tissue are able to differentiate into chondrocytes, osteocytes, adipocytes, and nerve cells (KR 10-1327076).

Meanwhile, rheumatoid arthritis is an autoimmune disease, and a chronic inflammatory disease associated with the chronic inflammation of the joints. Frequently, inflammation spreads to tissue around a joint and different organs. Generally, rheumatoid arthritis is a progressive disease that can lead to joint destruction and dysfunction, and joint inflammation associated with rheumatoid arthritis leads to the swelling, pain, stiffness, and redness of a joint. The joint inflammation associated with rheumatoid arthritis may also occur in tissues around joints (tendons, ligaments, and muscles). In some patients with rheumatoid arthritis, chronic inflammation destroys cartilage, bone, and ligaments, causing joint deformity. Damage to joints may occur early in the disease and may be progressive. Progressive joint damage does not necessarily correlate with the degree of pain, stiffness, or swelling in the joint.

Various clinical treatment methods for rheumatoid arthritis are being developed, and divided into general conservative therapy, drug therapy, and surgical therapy. Generally, the treatment of rheumatoid arthritis is aimed at a return to normal life by suppressing pain and inflammation and minimizing functional loss of joints because the disease causes joint pain, joint deformation, and dysfunction, which are caused by chronic arthritis.

Among the currently used treatment methods for rheumatoid arthritis, drug therapy is insufficient because it does not effectively suppress joint destruction even when three or more drugs are combined, and has side effects such as infection, gastrointestinal bleeding or perforation, deterioration of kidney and liver functions, osteoporosis, and Cushing's syndrome, and a problem of high cost.

Accordingly, to minimize permanent loss of joint function or serious side effects and improve the quality of life of patients, there is a need for the development of cell therapy for rheumatoid arthritis using stem cells.

### DISCLOSURE

### Technical Problem

While studying to develop a novel therapeutic agent effective in rheumatoid arthritis, the inventors experimentally confirmed that human nasal inferior turbinate-derived stem cells (hNTSCs) in which the expression of a hyaluronan synthase 2 (HAS2), C-X-C motif chemokine ligand 1 (CXCL1) or keratin associated protein 1-5 (KRTAP1-5) gene increases, or the expression of a glutathione S-transferase theta-2B (GSTT2B) or complement C4B (C4B) gene decreases have a therapeutic effect on rheumatoid arthritis, and the present invention was completed.

Therefore, the present invention is directed to providing a pharmaceutical composition for preventing or treating rheumatoid arthritis, which comprises stem cells as an active ingredient, wherein the stem cells have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof.

In addition, the present invention is directed to providing a cell therapeutic agent for treating rheumatoid arthritis, which comprises stem cells as an active ingredient, wherein the stem cells have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof.

In addition, the present invention is directed to providing a method of selecting stem cells for treating rheumatoid arthritis, which comprises measuring an expression or activity level of one or more proteins selected from the group consisting of HAS2, CXCL1, KRTAP1-5, GSTT2B, and C4B, or mRNA thereof in stem cells.

However, technical problems to be solved in the present invention are not limited to the above-described problems, and other problems which are not described herein will be fully understood by those of ordinary skill in the art from the following descriptions.

### Technical Solution

To achieve the purposes of the present invention, the present invention provides a pharmaceutical composition, which comprises stem cells as an active ingredient, wherein the stem cells have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof.

In addition, the present invention provides a cell therapeutic agent for treating rheumatoid arthritis, which comprises stem cells as an active ingredient, wherein the stem cells have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof. In one embodiment of the present invention, the stem cells may be hNTSCs, but the present invention is not limited thereto.

In another embodiment of the present invention, the rheumatoid arthritis may be collagen-induced arthritis, but the present invention is not limited thereto.

In addition, the present invention provides a method of selecting stem cells for treating rheumatoid arthritis, which comprises measuring an expression or activity level of one or more proteins selected from the group consisting of HAS2, CXCL1, and KRTAP1-5, GSTT2B and C4B, or mRNA thereof in stem cells.

In one embodiment of the present invention, the method may further comprise selecting stem cells as stem cells for treating rheumatoid arthritis when the expression or activity of one or more proteins selected from the group consisting of HAS2, CXCL1, and KRTAP1-5, or mRNA thereof increases; or when the expression or activity of one or more proteins selected from the group consisting of GSTT2B and C4B, or mRNA thereof decreases, but the present invention is not limited thereto.

In another embodiment of the present invention, the stem cells may be hNTSCs, but the present invention is not limited thereto.

In still another embodiment of the present invention, the expression level of the protein may be measured by one or more methods selected from the group consisting of western blotting, ELISA, radioimmunoassay, radioimmunodiffusion, Ouchterlony immunodiffusion, Rocket immunoelectrophoresis, immunohistostaining, immunoprecipitation assay, complement fixation assay, mass spectrometry, FACS, and a protein chip, but the present invention is not limited thereto.

In yet another embodiment of the present invention, the mRNA expression level may be measured by one or more methods selected from the group consisting of RT-PCR, RNase protection assay, Northern blotting, Southern blotting, in situ hybridization, and DNA chip, but the present invention is not limited thereto.

In addition, the present invention provides a method of preventing or treating rheumatoid arthritis, which comprises administering a pharmaceutical composition or cell therapeutic agent according to the present invention to a subject in need thereof.

In addition, the present invention provides a use of stem cells that have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof to prevent or treat rheumatoid arthritis.

In addition, the present invention provides a use of stem cells that have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof to produce a drug used for treatment of rheumatoid arthritis.

### Advantageous Effects

The present inventors confirmed that, in human nasal inferior turbinate-derived stem cells exhibiting a therapeutic effect on arthritis, specifically, the expression of a hyaluronan synthase 2 (HAS2), C-X-C motif chemokine ligand 1 (CXCL1) or keratin associated protein 1-5 (KRTAP1-5) gene increases, or the expression of a glutathione S-transferase theta-2B (GSTT2B) or complement C4B (C4B) gene decreases. Accordingly, by using the above fact, it is expected that only stem cells with a therapeutic effect can be selected and used for rheumatoid arthritis treatment, thereby improving the effect of treating rheumatoid arthritis, and furthermore, a method of selecting stem cells used in an agent for treating rheumatoid arthritis can be provided.

### DESCRIPTION OF DRAWINGS

FIGS. 1A to 1C show the results confirming the arthritis treatment effects per human nasal inferior turbinate-derived stem cell line in collagen-induced arthritis animal models (*P<0.05, **P<0.01, ***P<0.001, and hereinafter, n.s. means not significant).

FIG. 2 shows the heatmap results obtained by analyzing genes differentially expressed in human nasal inferior turbinate-derived stem cells with an arthritis treatment effect by a microarray (Y #cell line no.: effective, N #cell line no.: not effective, G: group).

FIG. 3 shows the results of comparing the expression levels of 14 types of candidate genes comprising hyaluronan synthase 2 (HAS2), C-X-C motif chemokine ligand 1 (CXCL1), keratin associated protein 1-5 (KRTAP1-5), glutathione S-transferase theta-2B (GSTT2B), and complement C4B (C4B), which are differentially expressed in human nasal inferior turbinate-derived stem cells having an arthritis treatment effect between cell lines effective in arthritis treatment and ineffective cell lines.

FIG. 4 shows the results of comparing the relative expression levels of mRNA of 14 types of candidate genes comprising HAS2, CXCL1, KRTAP1-5, GSTT2B, and C4B, which are differentially expressed in human nasal inferior turbinate-derived stem cells with an arthritis treatment effect between cell lines effective in arthritis treatment and ineffective cell lines.

### MODES OF THE INVENTION

The present inventors confirmed that the expression of a hyaluronan synthase 2 (HAS2), C-X-C motif chemokine ligand 1 (CXCL1) or keratin associated protein 1-(KRTAP1-5) gene specifically increases, or the expression of a glutathione S-transferase theta-2B (GSTT2B) or complement C4B (C4B) gene specifically decreases in human nasal inferior turbinate-derived stem cells having an effect of preventing or treating rheumatoid arthritis, and the present invention was completed.

Therefore, the present invention provides a pharmaceutical composition for preventing or treating rheumatoid arthritis and a cell therapeutic agent for treating rheumatoid arthritis, each of which comprises stem cells that have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof as an active ingredient.

Hereinafter, the present invention will be described in detail.

The present invention provides a pharmaceutical composition for preventing or treating rheumatoid arthritis, which comprises stem cells as an active ingredient.

The term “active ingredient” used herein refers to a component that can exhibit desired activity alone or exhibit desired activity with a carrier that is not active itself.

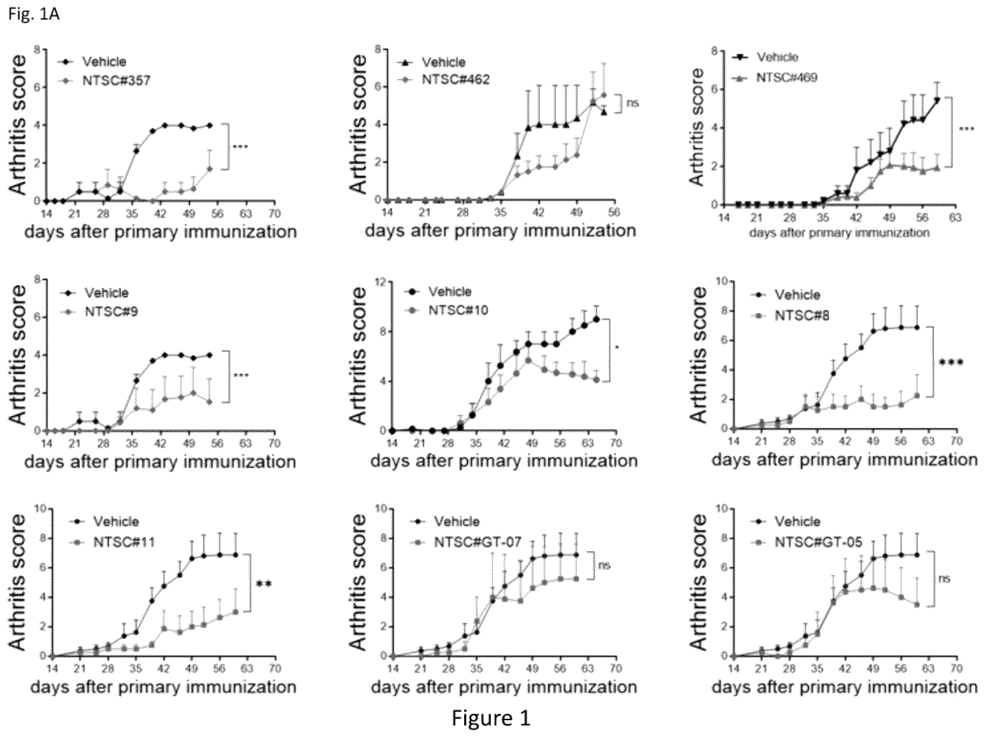

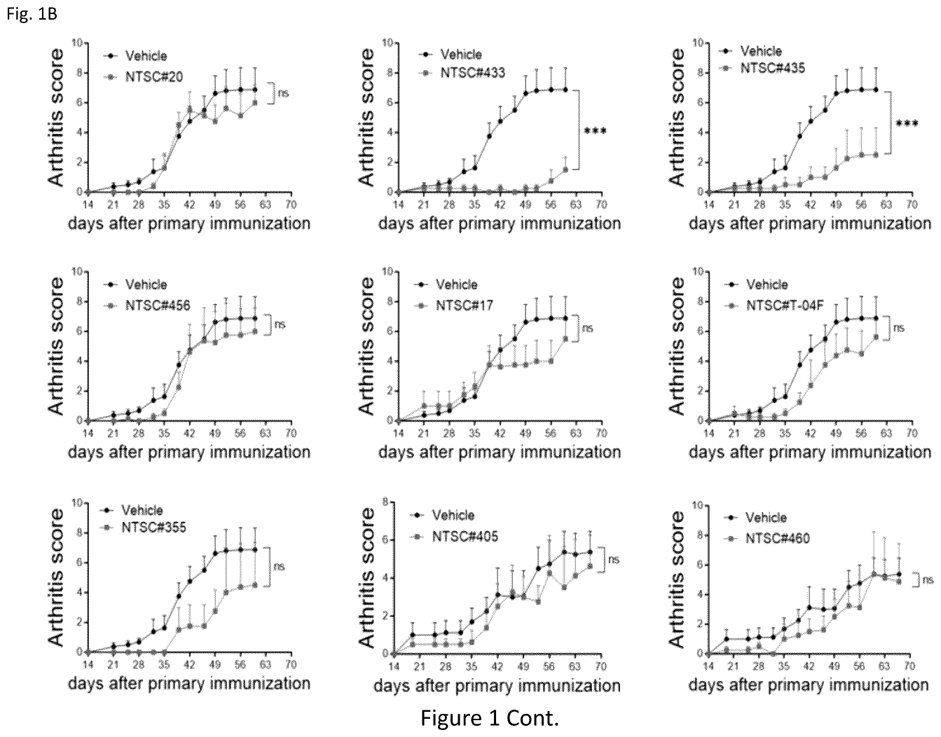

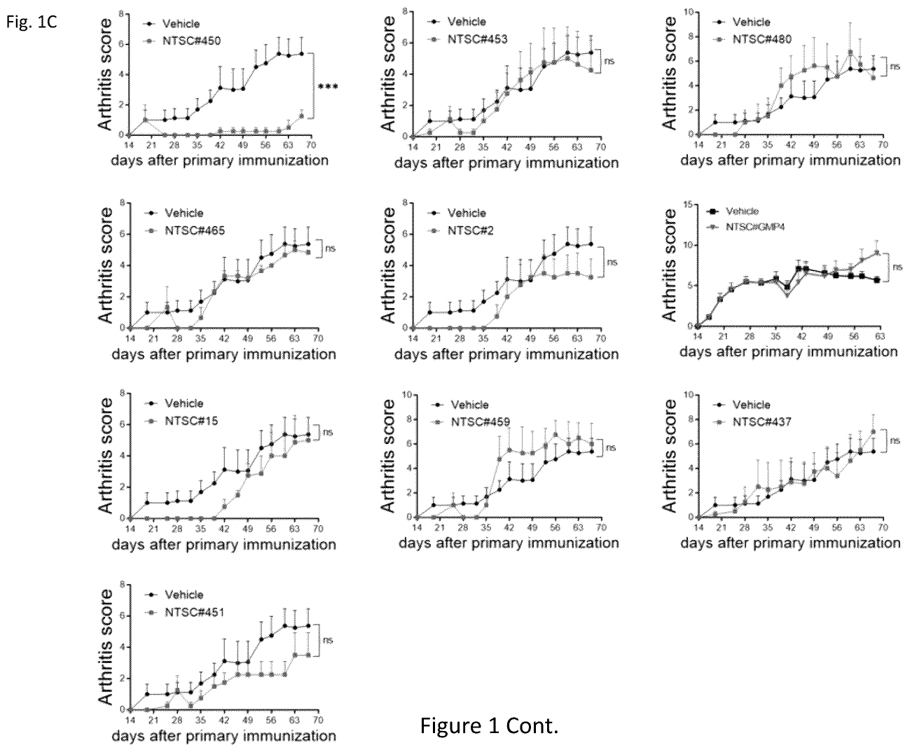

In one embodiment of the present invention, as the result of administering 28 human nasal inferior turbinate-derived stem cell lines to collagen-induced arthritis animal models, it was confirmed that there are stem cell lines that are not effective in arthritis treatment (refer to FIGS. 1A to 1C and Example 3).

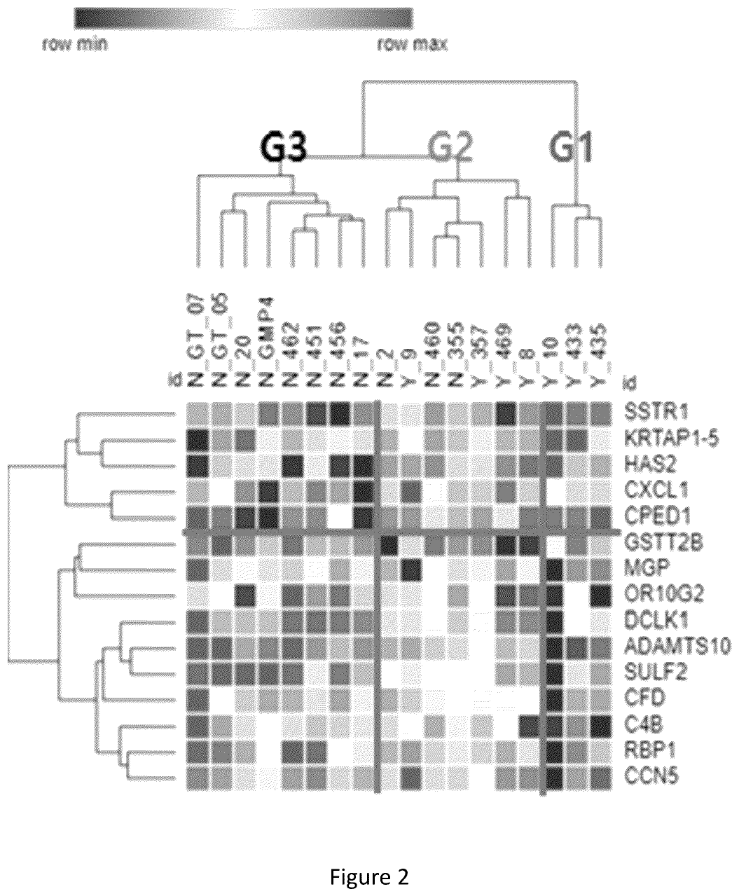

In another embodiment of the present invention, 14 types of candidate genes whose expression is specifically increased or decreased in cell lines exhibiting an effect in arthritis treatment were selected through microarray analysis (refer to FIG. 2 and Example 4).

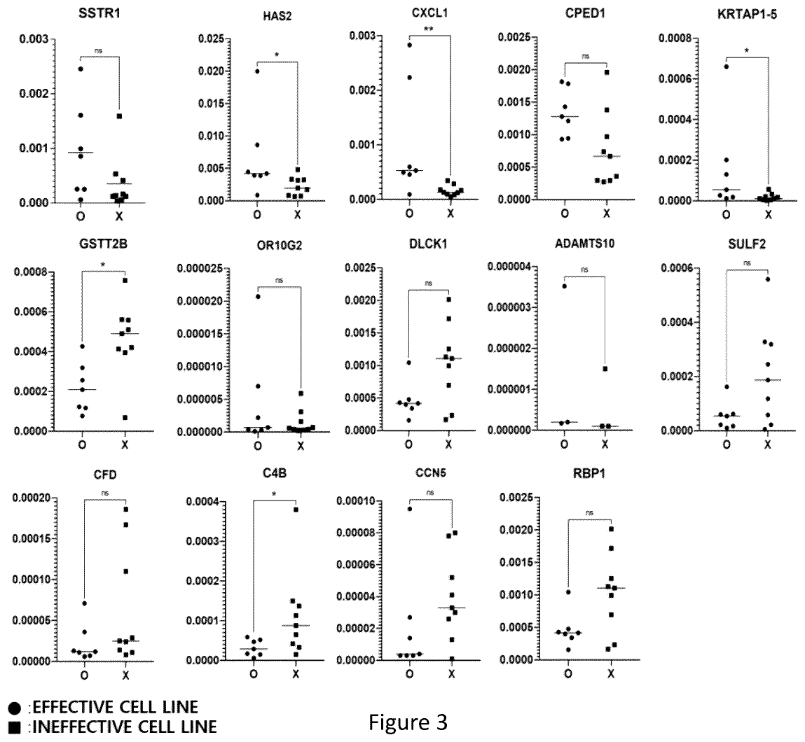

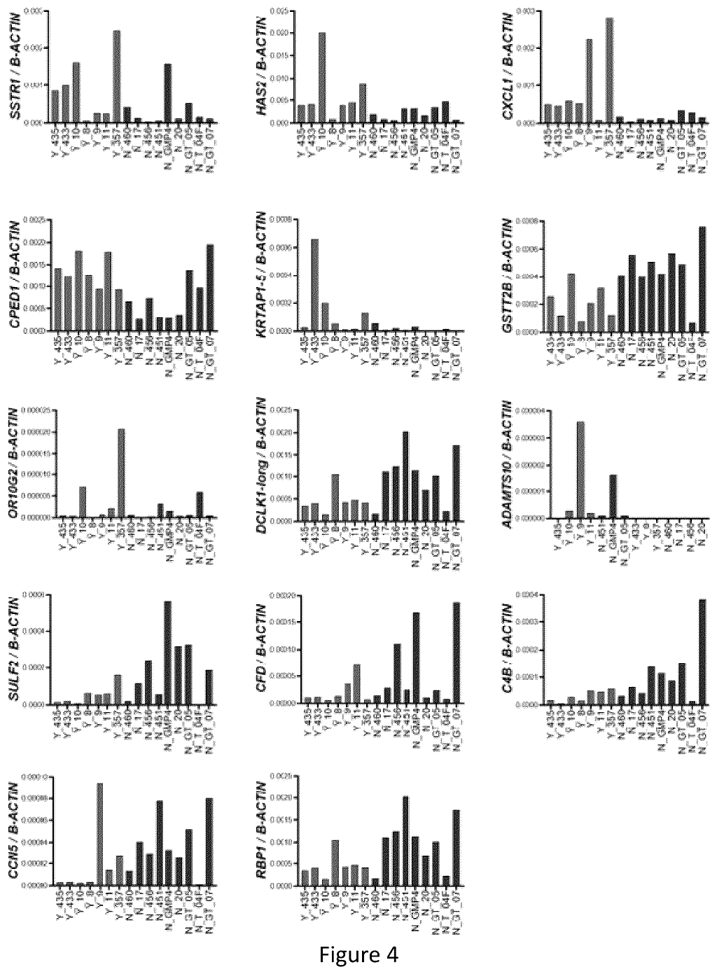

In still another embodiment of the present invention, as the result of verifying the expression of 14 types of selected candidate genes, it was confirmed that the expression of a HAS2, CXCL1, or KRTAP1-5 gene significantly increases, or the expression of a GSTT2B or C4B gene significantly decreases in cell lines effective in arthritis treatment (refer to FIGS. 3 and 4, and Example 5).

Accordingly, the stem cells may be characterized in that the expression or activity of one or more proteins selected from the group consisting of HAS2, CXCL1, and KRTAP1-5, or mRNA thereof is increased; or
the expression or activity of one or more proteins selected from the group consisting of GSTT2B and C4B or mRNA thereof is decreased, and specifically, when compared with an ineffective stem cell line, the expression or activity of one or more proteins selected from the group consisting of HAS2, CXCL1, and KRTAP1-5, or mRNA thereof is increased; or
the expression or activity of one or more proteins selected from the group consisting of GSTT2B and C4B, or mRNA thereof is decreased, but the present invention is not limited thereto.

The DNA sequences or amino acid sequences of HAS2, CXCL1, KRTAP1-5, GSTT2B, and C4B of the present invention are known, and may be obtained from a known database such as NCBI GenBank.

The term “effective” used herein means that a drug is effective in arthritis, so it inhibits or delays the onset of arthritis or alleviates or reduces the symptoms of arthritis, and in contrast, the “ineffective” used herein means that a drug is not effective in arthritis or has little effect, so it does not inhibit or delay the onset of arthritis or alleviate or reduce the symptoms of arthritis.

In the present invention, the stem cells may be human nasal inferior turbinate-derived stem cells (hNTSCs), which are specifically isolated from nasal inferior turbinate tissue obtained in the process of performing human nasal inferior turbinate resection.

The term “stem cell” used herein refers to a cell that becomes the basis of a cell or tissue constituting an individual, repeatedly divides to enable self-renewal, and has multipotency capable of differentiating into cells with specific functions according to an environment. It is produced from all tissues during fetal development, and even during adulthood, it is found in some tissues in which cells are actively replaced, such as bone marrow and epithelial tissue. Stem cells are divided into totipotent stem cells, which are formed when the fertilized egg begins to divide for the first time, pluripotent stem cells in the inner membrane of the blastocyst formed by continuous division of these cells, and multipotent stem cells present in mature tissues and organs according to the type of differentiable cells. Here, multipotent stem cells are cells that can differentiate only into cells specific to tissues and organs comprising these cells and are involved in growing and developing each tissue or organ in the prenatal, neonatal, and adult stages, maintaining the homeostasis of adult tissue, and inducing regeneration in the case of tissue damage. These tissue-specific multipotent cells are collectively called adult stem cells.

Among adult stem cells, bone marrow-derived stem cells and adipose stem cells have disadvantages in that a surgery for acquiring the cells is accompanied by excruciating pain and takes a lot of time, the amount of acquired stem cells is very small, lots of time and money are consumed in the process of culturing a clinically sufficient amount, and the risk of infection and cell loss is high. On the other hand, human nasal inferior turbinate-derived stem cells have advantages in that a surgery for acquiring the cells has very little bleeding and pain and takes less time, the stem cells can be continuously obtained through recycling of stem cells isolated from discarded nasal inferior turbinate tissue during nasal inferior turbinate surgery (rhinitis surgery) most frequently performed in the otolaryngology field, and the proliferative ability of the stem cells is higher than those of the bone marrow-derived and adipose stem cells.

In addition, according to one embodiment of the present invention, the rheumatoid arthritis may be collagen-induced arthritis, but the present invention is not limited thereto (refer to Examples 1-2).

The term “protein” used herein is interchangeably used with a “polypeptide” or “peptide,” and refers to, for example, a polymer of amino acid residues as generally found in a protein in a natural state. The “mRNA” used herein refers to RNA that delivers genetic information (gene-specific base sequence) to a ribosome specifying an amino acid sequence from a specific gene in a protein synthesis process.

The term “pharmaceutical composition” used herein is prepared to prevent or treat a disease and may be used by being formulated in various forms according to conventional methods. For example, the pharmaceutical composition may be formulated in oral forms such as a powder, a granule, a tablet, a capsule, a suspension, an emulsion, and a syrup, or in the forms of an external preparation, a suppository, and a sterile injection solution.

The pharmaceutical composition according to the present invention may further comprise suitable carrier, excipient and diluent, which are conventionally used in preparation of a pharmaceutical composition. For example, the excipient may be one or more selected from the group consisting of a diluent, a binder, a disintegrant, a lubricant, an adsorbent, a humectant, a film-coating material, and a controlled-release additive.

The pharmaceutical composition according to the present invention may be formulated in the form of a powder, a granule, a sustained-release granule, an enteric granule, a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or an external preparation such as an aerosol according to a conventional method, and the external preparation may be formulated in a cream, a gel, a patch, a spray, an ointment, a plaster, a lotion, a liniment, a paste or a cataplasma.

The carrier, excipient and diluent which may be comprised in the pharmaceutical composition according to the present invention may comprise lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

The pharmaceutical composition may be formulated with a diluent or an excipient such as a filler, a thickening agent, a binder, a wetting agent, a disintegrant, and a surfactant, which are commonly used.

As additives for a tablet, powder, granule, capsule, pill and troche, excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, carboxymethylcellulose, sodium carboxymethylcellulose, kaolin, urea, colloidal silica gel, hydroxypropyl starch, hydroxypropyl methyl cellulose (HPMC) 1928, HPMC 2208, HPMC 2906, HPMC 2910, propylene glycol, casein, calcium lactate and Primojel; binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol and polyvinylpyrrolidone; disintegrants such as hydroxypropylmethylcellulose, corn starch, agar powder, methylcellulose, bentonite, hydroxypropyl starch, sodium carboxymethylcellulose, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, sucrose, magnesium aluminum silicate, a di-sorbitol solution and light anhydrous silicic acid; and lubricants such as calcium stearate, magnesium stearate, stearic acid, hydrogenated vegetable oil, talc, limestone kaolin, petrolatum, sodium stearate, cacao butter, sodium salicylate, magnesium salicylate, polyethylene glycol (PEG) 4000 and, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dileucine and light anhydrous silicic acid may be used.

As an additive for the liquid formulation according to the present invention, water, diluted hydrochloric acid, diluted sulfuric acid, sodium citrate, sucrose monostearate, polyoxyethylene sorbitol fatty acid ester (Tween ester), polyoxyethylene monoalkyl ether, lanolin ether, lanolin ester, acetic acid, hydrochloric acid, aqueous ammonia, ammonium carbonate, potassium hydroxide, sodium hydroxide, prolamine, polyvinylpyrrolidone, ethyl cellulose, or sodium carboxymethylcellulose may be used.

In the syrup according to the present invention, a solution of white sugar, other sugars, or a sweetener may be used, and an aromatic, a coloring agent, a preservative, a stabilizer, a suspending agent, an emulsifier, or a thickening agent may be used as necessary.

In the emulsion according to the present invention, distilled water may be used, and an emulsifier, a preservative, a stabilizer, or a preservative may be used as necessary.

In the suspension according to the present invention, a suspending agent such as acacia, tragacanth, methyl cellulose, carboxymethyl cellulose, sodium carboxymethylcellulose, microcrystalline cellulose, sodium alginate, hydroxypropylmethyl cellulose, HPMC 1828, HPMC 2906, or HPMC 2910 may be used, and a surfactant, a preservative, a stabilizer, a coloring agent, and a fragrance may be used as necessary.

In an injection according to the present invention, a solvent such as injectable sterile water, 0.9% sodium chloride for injection, Ringer's solution, a dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride; a stabilizer such as sodium bisulfate (NaHSO3), carbon dioxide gas, sodium metabisulfite (Na₂S₂O₃), sodium sulfite (Na₂SO₃), nitrogen gas (N₂) or ethylenediaminetetracetic acid; an antioxidant such as sodium bisulfide 0.1%, sodium formaldehyde sulfoxylate, thiourea, disodium ethylenediaminetetraacetate or acetone sodium bisulfite; a pain-relief agent such as benzyl alcohol, chlorobutanol, procaine hydrochloride, glucose or calcium gluconate; or a suspending agent such as sodium CMC, sodium alginate, Tween 80 or aluminum monostearate may be used.

As a suppository according to the present invention, a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M, Fs), or a Tegester triglyeride base (TG-95, MA, 57) may be used.

A solid formulation for oral administration may be a tablet, a pill, a powder, a granule or a capsule, and such a solid formulation may be prepared by mixing at least one of excipients, for example, starch, calcium carbonate, sucrose, lactose and gelatin, with the active ingredient. Also, in addition to the simple excipient, lubricants such as magnesium stearate and talc may also be used.

As a liquid formulation for oral administration, a suspension, a liquid for internal use, an emulsion, or a syrup may be used, and a generally-used simple diluent such as water or liquid paraffin, as well as various types of excipients, for example, a wetting agent, a sweetener, a fragrance and a preservative may be comprised. A formulation for parenteral administration may be a sterilized aqueous solution, a non-aqueous solvent, a suspension, an emulsion, a lyophilizing agent or a suppository. As the non-aqueous solvent or suspension, propylene glycol, polyethylene glycol, a vegetable oil such as olive oil, or an injectable ester such as ethyl oleate may be used.

The composition according to the present invention is administered at a pharmaceutically effective amount. In the present invention, the “pharmaceutically effective amount” used herein refers to an amount sufficient for treating a disease at a reasonable benefit/risk ratio applicable for medical treatment, and an effective dosage may be determined by parameters comprising a type of a patient's disease, severity, drug activity, sensitivity to a drug, administration time, an administration route and an excretion rate, the duration of treatment and drugs simultaneously used, and other parameters well known in the medical field.

The pharmaceutical composition of the present invention may be administered separately or in combination with other therapeutic agents and may be sequentially or simultaneously administered with a conventional therapeutic agent, or administered in a single or multiple dose(s). In consideration of all the above-mentioned parameters, it is important to achieve the maximum effect with the minimum dose without a side effect, and such a dose may be easily determined by one of ordinary skill in the art.

The pharmaceutical composition of the present invention may be administered to a subject via various routes. All administration routes may be expected, and the pharmaceutical composition of the present invention may be administered by, for example, oral administration, subcutaneous injection, intraperitoneal administration, intravenous, intramuscular or intrathecal injection, sublingual administration, buccal administration, rectal insertion, vaginal insertion, ocular administration, ear administration, nasal administration, inhalation, spraying through the mouth or nose, skin administration, or transdermal administration.

In addition, in another aspect of the present invention, the present invention provides a cell therapeutic agent for rheumatoid arthritis, which comprises stem cells as an active ingredient, wherein the stem cells have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof.

The term “cell therapeutic agent” used herein refers to cells and tissue used for treatment, diagnosis and prevention and prepared by isolation from a human, culture, and special manipulation, and refers to a pharmaceutical product used for treatment, diagnosis and prevention through a series of actions of in vitro proliferating or selecting homogeneous or heterogeneous cells to restore the function of cells or tissue, or as another method, changing the biological characteristics of cells. The cell therapeutic agent is largely classified into a somatic cell therapeutic agent and a stem cell therapeutic agent according to the degree of cell differentiation, and the present invention relates to, particularly, a stem cell therapeutic agent.

In addition, the present invention provides a method of preventing or treating rheumatoid arthritis, which comprises administering the pharmaceutical composition or cell therapeutic agent according to the present invention to a subject in need thereof.

In addition, the present invention provides a use of stem cells that have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof to prevent or treat rheumatoid arthritis.

In addition, the present invention provides a use of stem cells that have increased expression or activity of one or more proteins selected from the group consisting of HAS2, CXCL1, and KRTAP1-5, or mRNA thereof; or decreased expression or activity of one or more proteins selected from the group consisting of GSTT2B and C4B, or mRNA thereof to produce a drug used for treatment of rheumatoid arthritis.

The term “subject” used herein refers to a target in need of treatment, and more specifically, a mammal such as a human or a non-human primate, a mouse, a rat, a dog, a cat, a horse, or a cow.

The “administration” used herein refers to providing the composition of the present invention to a subject by any suitable method.

The term “prevention” used herein refers to all actions of inhibiting rheumatoid arthritis or delaying the occurrence thereof by administration of the pharmaceutical composition or cell therapeutic agent of the present invention.

The term “treatment” used herein refers to all actions involved in alleviating or beneficially changing symptoms of rheumatoid arthritis by administration of the pharmaceutical composition or cell therapeutic agent according to the present invention.

The term “improvement” used herein refers to all actions of reducing parameters associated with rheumatoid arthritis, for example, the severity of a symptom, by administration of the pharmaceutical composition or cell therapeutic agent according to the present invention.

Another aspect of the present invention provides a method of selecting stem cells for treating rheumatoid arthritis, which comprises a method of selecting stem cells for treating rheumatoid arthritis, which comprises measuring an expression or activity level of one or more proteins selected from HAS2, CXCL1, and KRTAP1-5, GSTT2B and C4B from stem cells, or mRNA thereof in stem cells.

In the present invention, the method may further comprise selecting stem cells as stem cells for treating rheumatoid arthritis when the expression or activity of one or more proteins selected from HAS2, CXCL1, and KRTAP1-5, or mRNA thereof increases; or

when the expression or activity of one or more proteins selected from GSTT2B and C4B, or mRNA thereof decreases, and specifically, compared with an ineffective stem cell line, when the expression or activity of one or more proteins selected from the group consisting of HAS2, CXCL1, and KRTAP1-5, or mRNA thereof increases; or when the expression or activity of one or more proteins selected from the group consisting of GSTT2B and C4B, or mRNA thereof decreases, but the present invention is not limited thereto.

In addition, according to the present invention, the method may further comprise extracting DNA or a protein from stem cells before measuring expression or activity levels of the protein or mRNA thereof, and the DNA or protein extraction may be performed by a method known in the art, and the method is not limited as long as it can extract DNA or a protein from stem cells.

In the present invention, the protein expression level may be measured by one or more methods selected from the group consisting of western blotting, ELISA, radioimmunoassay, radioimmunodiffusion, Ouchterlony immunodiffusion, Rocket immunoelectrophoresis, immunohistostaining, immunoprecipitation assay, complement fixation assay, mass spectrometry, FACS, and a protein chip, but the present invention is not limited thereto.

In the present invention, the mRNA expression level may be measured by one or more methods selected from the group consisting of RT-PCR, RNase protection assay, Northern blotting, Southern blotting, in situ hybridization, and a DNA chip, but the present invention is not limited thereto.

Hereinafter, to help in understanding the present invention, exemplary examples will be suggested. However, the following examples are merely provided to understand the present invention more easily, and not to limit the present invention.

### EXAMPLES

### Example 1. Materials and Methods

1-1. Culture of Human Nasal Inferior Turbinate-Derived Stem Cells (hNTSCs)

Human nasal inferior turbinate tissue used herein was tissue obtained in the process of performing human nasal inferior turbinate resection and used with the patient's consent. Immediately after collecting the nasal inferior turbinate tissue, fibroblasts were isolated by washing 3 to 5 times with physiological saline comprising gentamicin (Kukje Pharma Co. Ltd., Seongnam, Korea).

To isolate hNTSCs, nasal inferior turbinate tissue removed during surgery was refrigerated at 4° C., and the tissue was washed three times with an antibiotic-antifungal solution (Gibco, Gaithersberg, MD) at room temperature. After washing again with neutral phosphate buffered saline (PBS) three times, it was cut into small pieces of 1 mm³ using small surgical scissors.

The hNTSCs were put on a 100-mm culture plate, covered with a sterilized slide glass to make the cells attached to the culture plate, Dulbecco's Modified Eagle's Media (DMEM) supplemented with 10% fetal bovine serum (FBS) was added, and the hNTSCs were cultured in an incubator at 37° C. in a 5% CO₂ environment. After culturing for 2 to 3 weeks, the slide glass was removed, cells floating in the culture were washed and discarded, hNTSCs attached to the bottom of the culture plate were detached from the bottom using trypsin and then sub-cultured for 6 passages.

1-2. Establishment of Collagen-Induced Arthritis and Administration of hNTSCs

7-week-old make DBA1/J(JAX™) mice were acclimated to the environment of an animal laboratory for 1 week. The conditions of an animal breeding room were 22.0±2° C., illumination at 200 to 300 Lux for 12 hours per day, and light was blocked for 12 hours. 100 μg of type 2 collagen (Chondrex) and a complete Freund's adjuvant (CFA; Chondrex) were mixed in a ratio of 1:1 (w/v) and then intradermally injected into the tail of a DBA1/J mouse for a first immunization injection, and two weeks later, 100 μg of type 2 collagen and an incomplete Freund's adjuvant were mixed in a ratio of 1:1 (v/v) and injected intradermally into the tail for a second immunization injection. Between 5 to 6 weeks after the first immunization injection, hNTSCs were prepared at 1×10⁶ cells/100 μl/mouse and injected into the tail vein three times, and for a control, a phosphate-buffered saline (PBS) solution was injected into the same site at 100 μl/mouse.

1-3. Evaluation of Arthritis

After confirming that erythema gradually appeared on the 14^{th} day after the first immunization injection and arthritis developed, cages were redistributed so that the mean arthritis index for each group was consistent, and then a PBS solution was injected into a control, and hNTSCs were injected into an experimental group.

Visual observation of arthritic lesions was performed using the following scores based on the reference (Barnett M L, Kremer J M, St Clair E W, Clegg D O, Furst D, Weisman M, et al. Treatment of rheumatoid arthritis with oral type II collagen. Results of a multicenter, double-blind, placebo-controlled trial. Arthritis Rheum 1998; 41:290-7).
0 points: No edema or swelling, 1 point: Mild swelling and redness localized to a foot or an ankle joint, 2 points: Mild swelling and redness from an ankle joint to a tarsal bone, 3 points: Moderate swelling and redness from an ankle joint to a tarsal bone, and 4 points: swelling and redness from an ankle to the entire leg and joint stiffness.

Accordingly, the highest score of the arthritis lesion is 12 points for one mouse, excluding the score of a leg subjected to the second immunization injection, the arthritis lesions were observed three times from the time of the first immunization injection to the 11^{th} week, and the evaluation data was prepared by three persons who were not related to the experiment. The average arthritis indexes were compared between the PBS-administered group and the NTSC-administered group.

1-4. Microarray Analysis

Microarray analysis was performed by Macrogen Co. using an Agilent Human GE 8×60K V3 chip (Agilent) according to the manufacturer's instructions.

1-5. mRNA Analysis of hNTSCs

hNTSCs were treated with 1 mL of RNA iso (Takara) to extract total RNA. An extraction method followed the manufacturer's recommendations. The extracted RNA was quantified using a Nanodrop device, and 2 μg RNA was reverse-transcribed into cDNA using a Dyne First Strand cDNA Synthesis Kit (Dyne Bio). 1 μl of cDNA was put into a 0.2 ml tube, 1 μl Probe (4 pmol/μl), 1 μl sense primer (10 pmol/μl), 1 μl antisense primer (10 pmol/μl), 10 μl Dyne Ab qPCR 2× PreMIX (Dyne Bio), and 4 μl RNase free water (Dyne Bio) were mixed, and real-time amplification was performed in a LightCycler96 device (Roche) under an annealing condition of 60° C. For relative quantification of each target mRNA, a beta-actin gene was amplified in the same way, and all the used primers and probes are shown in Table 1. For relative quantification, as in a conventional method, a Cq level was obtained by automatically calculating the time to start amplification of an mRNA concentration after real-time amplification, and the Cq level of the target gene was subtracted by the Cq level of beta-actin to obtain a delta Cq (dCq) value. This was converted into a 2^{-dCq} value and compared.

TABLE 1

| Gene | 5′-3′ | Sequence |
|---|---|---|
| SSTR1 | Sense | TGAGTCAGCTGTCGGTCATC (SEQ ID NO: 1) |
| | Antisense | GGAAAGAGCGCTTGAAGTTG (SEQ ID NO: 2) |
| | Probe | 5′FAM-TATGCCAACAGCTGCGCCAACCCCA-3′BHQ1 (SEQ |
| | | ID NO: 3) |
| HAS2 | Sense | CTGGGACGAAGTGTGGATTATG (SEQ ID NO: 4) |
| | Antisense | GATGAGGCTGGGTCAAGCAT (SEQ ID NO: 5) |
| | Probe | 5′CY5-AGGTTTGTGATTCAGACACTATGCTTGACC-3′BHQ3 |
| | | (SEQ ID NO: 6) |
| CXCL1 | Sense | TCCTGCATCCCCCATAGTTA (SEQ ID NO: 7) |
| | Antisense | CTTCAGGAACAGCCACCAGT (SEQ ID NO: 8) |
| | Probe | 5′TexasRed-CTTCCTCCTCCCTTCTGGTCAGTTG-3′BHQ2 |
| | | (SEQ ID NO: 9) |
| CPED1 | Sense | GTGAAACATCTACTCTGGGACC (SEQ ID NO: 10) |
| | Antisense | TTAAATGCTCGTGTACCTGGAG (SEQ ID NO: 11) |
| | Probe | 5′HEX-TTGGTTATGGCAGTTTCATGTACCCTGT-3′BHQ1 |
| | | (SEQ ID NO: 12) |
| KRTAP1-5 | Sense | TGAGCCCACTTGCTGAAAG (SEQ ID NO: 13) |
| | Antisense | TGTAGCATTTCTGTGTCCCC (SEQ ID NO: 14) |
| | Probe | 5′HEX-ACTGTTCATCCCTTGACCACCTCTG-3′BHQ1 (SEQ ID |
| | | NO: 15) |
| GSTT2B | Sense | GCAGCCGGTGGCTCTC (SEQ ID NO: 16) |
| | Antisense | AAGATGATGCTGTGGGCCTC (SEQ ID NO: 17) |
| | Probe | 5′FAM-ATGAACTGTTTGAGGGACGGCCACGA-3′BHQ1 |
| | | (SEQ ID NO: 18) |
| OR10G2 | Sense | TCCTCAGTCACCGTTCCTCT (SEQ ID NO: 19) |
| | Antisense | AACCTCCCATTCATGAGCAC (SEQ ID NO: 20) |
| | probe | 5′CY5-CCGCCAGTTGCTTCATGTTAATTCTGCTC-3′BHQ3 |
| | | (SEQ ID NO: 21) |
| DLCK1 | Sense | GACATGGAGCTGGAGCACTT (SEQ ID NO: 22) |
| | Antisense | CTTCTTCTCGGAGCTGAGCG (SEQ ID NO: 23) |
| | Probe | 5′CY5-TCACTGGCCACTGCCAAAGGAAGCC-3′BHQ3 (SEQ |
| | | ID NO: 24) |
| ADAMTS10 | Sense | CTCATGTTCGAGGTCACGCA (SEQ ID NO: 25) |
| | Antisense | CACTTTGTAGAAGAGGCGGGA (SEQ ID NO: 26) |
| | Probe | 5′FAM-ATGAGTTCCTGTCCAGTCTGGAGAGC-3′BHQ1 (SEQ |
| | | ID NO: 27) |
| SULF2 | Sense | TGAAAGGCAGGTTTCAGAGGG (SEQ ID NO: 28) |
| | Antisense | GTGGTCACGAAGGCGTTGAT (SEQ ID NO: 29) |
| | Probe | 5′HEX-AGGAACATCCGCCCCAACATCATCCT-3′BHQ1 (SEQ |
| | | ID NO: 30) |
| CFD | Sense | GACACCATCGACCACGAC (SEQ ID NO: 31) |
| | Antisense | GTTGACTATGCCCCAGCC (SEQ ID NO: 32) |
| | Probe | 5′CY5-AGCTGTCGGAGAAGGCCACAC-3′BHQ3 (SEQ ID NO: |
| | | 33) |
| MGP | Sense | CCCTTCATTAACAGGAGAAATGCAA (SEQ ID NO: 34) |
| | Antisense | GCGTAGCGTTCGCAAAGTC (SEQ ID NO: 35) |
| | Probe | 5′TexasRed-TCCCCTCAGCAGAGATGGAGAGCTAAA- |
| | | 3′BHQ2 (SEQ ID NO: 36) |
| C4B | Sense | GTGGCCTCCATCAACTCCTC (SEQ ID NO: 37) |
| | Antisense | TGTAAATGGGCTGGTCCGTC (SEQ ID NO: 38) |
| | Probe | 5′FAM-AGGTCCAGCTGGTGGCCCATTCG-3′BHQ1 (SEQ ID |
| | | NO: 39) |
| CCN5 | Sense | CCTCCTCTGCCTCCTCTCAA (SEQ ID NO: 40) |
| | Antisense | GACGTGGAGTTGGTCGCA (SEQ ID NO: 41) |
| | Probe | 5′HEX-ACCCAGCTGTGCCCGACACCATGT-3′BHQ1 (SEQ ID |
| | | NO: 42) |
| RBP1 | Sense | TTGCGCAAAATCGCCAACTT (SEQ ID NO: 43) |
| | Antisense | GCGGTCATCTATGCCTGTCA (SEQ ID NO: 44) |
| | Probe | 5′CY5-AAAGAGATCGTGCAGGACGGTGACCA-3′BHQ3 |
| | | (SEQ ID NO: 45) |
| β-actin | Sense | GGACTTCGAGCAAGAGATGG (SEQ ID NO: 46) |
| | Antisense | TGTGTTGGGGTACAGGTCTTTG (SEQ ID NO: 47) |
| | Probe | 5′TexasRed-TAAGGAGAATGGCCCAGTCCTCTCCCAA3- |
| | | BHQ2 (SEQ ID NO: 48) |

### Example 2. Preparation of Collagen-Induced Arthritis Animal Model and Administration of hNTSCs

100 μg of type 2 collagen (Chondrex) and a complete Freund's adjuvant were mixed in a ratio of 1:1 (w/v) and then intradermally injected into the tail of an 8-week-old male DBA1/J mouse for a first immunization injection, and two weeks later, 100 μg of type 2 collagen and a incomplete Freund's adjuvant were mixed in a ratio of 1:1 (w/v) and then intradermally injected into the tail of the mouse and then injected subcutaneously for a second immunization injection.

Between 5 to 6 weeks after the first immunization injection, 1×10⁶ hNTSCs were injected into the tail vein three times, and for a control, 100 μl of a PBS solution was injected and then the arthritis index was observed for an additional 5 weeks until the 11^{th} week.

### Example 3. Comparison of Arthritis Indexes in Collagen-Induced Arthritis Animal Models According to Administration of hNTSCs

To confirm the therapeutic effect of hNTSCs on rheumatoid arthritis, each of 28 stem cell lines was administered to a rheumatoid arthritis animal model by the method described in Example 1-3, and then arthritis was evaluated. All data was expressed as mean±SEM (***P<0.001).

As a result, as shown in FIGS. 1A to 1C, among 28 cell lines, there are 9 cell lines confirmed to be effective in treating rheumatoid arthritis by continuously delaying the progression of the onset of arthritis until the end of the experiment, which is significantly slower than the control, and 19 cell lines confirmed to be ineffective.

### Example 4. Microarray Analysis of Genes Differentially Expressed in hNTSCs Effective in Rheumatoid Arthritis Animal Models

To select a gene differentially expressed in a cell line effective in arthritis treatment, for 20 cell lines (however, in FIG. 2, #11 and #T-04F cell lines were omitted) out of 28 cell lines subjected to evaluation of their effectiveness in rheumatoid arthritis treatment in Example 3, microarray analysis was performed by the method described in Example 1-4.

As a result of microarray analysis, as shown in FIG. 2, 15 types of candidate genes that best represent the characteristics of effective and ineffective cell lines were selected. Since MGP among the selected 15 types of genes has a significantly different result value in a specific cell line, it was judged to be an outlier that distorts the results and was excluded from the final candidate genes, and 14 types of final candidate genes differentially expressed in cells effective in arthritis treatment were selected.

### Example 5. Validation of Expression of 14 Types of Candidate Genes Used for Selection of hNTSCs for Treating Rheumatoid Arthritis

The 14 types of candidate genes obtained in Example 4 were verified according to the method of Example 1-5, and 4 cell lines of the 20 cell lines of Example 4 were excluded from the result values because RNA degradation was suspected due to severe variation in beta-actin values.

As a result of verification of the expression of candidate genes using the result values of 16 cell lines (7 effective cell lines, and 9 ineffective cell lines), as shown in FIGS. 3 and 4, when comparing the effective cell line group and the ineffective cell line group among the 14 types of candidate genes selected in Example 4, it was confirmed that the genes that had significantly increased expression are HAS2, CXCL1, and KRTAP1-5, and the genes that had significantly decreased expression are GSTT2B and C4B.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

The present inventors confirmed that, in human nasal inferior turbinate-derived stem cells effective in treatment of arthritis, specifically, the expression of a hyaluronan synthase 2 (HAS2), C-X-C motif chemokine ligand 1 (CXCL1) or keratin associated protein 1-5 (KRTAP1-5) gene increases, or the expression of a glutathione S-transferase theta-2B (GSTT2B) or complement C4B (C4B) gene decreases. Accordingly, as only stem cells having a therapeutic effect are selected and used for treatment of rheumatoid arthritis using the above fact, it is expected to improve a rheumatoid arthritis treatment effect and further provide a method of selecting stem cells used in a rheumatoid arthritis therapeutic agent.

1. A pharmaceutical composition for treating rheumatoid arthritis, comprising stem cells as an active ingredient,
wherein the stem cells have increased expression or activity of one or more proteins selected from the group consisting of hyaluronan synthase 2 (HAS2), C-X-C motif chemokine ligand 1 (CXCL1), and keratin associated protein 1-5 (KRTAP1-5), or mRNA thereof; or
decreased expression or activity of one or more proteins selected from the group consisting of glutathione S-transferase theta-2B (GSTT2B) and complement C4B (C4B), or mRNA thereof.

2-3. (canceled)

4. A cell therapeutic agent for treating rheumatoid arthritis, comprising stem cells as an active ingredient,
wherein the stem cells have increased expression or activity of one or more proteins selected from the group consisting of hyaluronan synthase 2 (HAS2), C-X-C motif chemokine ligand 1 (CXCL1), and keratin associated protein 1-5 (KRTAP1-5), or mRNA thereof; or
decreased expression or activity of one or more proteins selected from the group consisting of glutathione S-transferase theta-2B (GSTT2B) and complement C4B (C4B), or mRNA thereof.

5. The cell therapeutic agent of claim 4, wherein the stem cells are human nasal turbinate-derived stem cells (hNTSCs).

6. The cell therapeutic agent of claim 4, wherein the rheumatoid arthritis is collagen-induced arthritis.

7. A method of selecting stem cells for treating rheumatoid arthritis, comprising:
measuring an expression or activity level of one or more proteins selected from the group consisting of hyaluronan synthase 2 (HAS2), C-X-C motif chemokine ligand 1 (CXCL1), keratin associated protein 1-5 (KRTAP1-5), glutathione S-transferase theta-2B (GSTT2B), and complement C4B (C4B), or mRNA thereof in stem cells.

8. The method of claim 7, further comprising:
selecting stem cells that have increased expression or activity of one or more proteins selected from the group consisting of hyaluronan synthase 2 (HAS2), C-X-C motif chemokine ligand 1 (CXCL1), and keratin associated protein 1-5 (KRTAP1-5), or mRNA thereof; or
decreased expression or activity of one or more proteins selected from the group consisting of glutathione S-transferase theta-2B (GSTT2B) and complement C4B (C4B), or mRNA thereof as stem cells for treating rheumatoid arthritis.

9. The method of claim 7, wherein the stem cells are human nasal inferior turbinate-derived stem cells (hNTSCs).

10. The method of claim 7, wherein the expression level of the protein is measured by one or more methods selected from the group consisting of western blotting, ELISA, radioimmunoassay, radioimmunodiffusion, Ouchterlony immunodiffusion, Rocket immunoelectrophoresis, immunohistostaining, immunoprecipitation assay, complement fixation assay, mass spectrometry, FACS, and a protein chip.

11. The method of claim 7, wherein the expression level of mRNA is measured by one or more methods selected from the group consisting of RT-PCR, RNase protection assay, Northern blotting, Southern blotting, in situ hybridization, and a DNA chip.

12. A method of treating rheumatoid arthritis, comprising:
administering the pharmaceutical composition of claim 1 or the cell therapeutic agent of any one of claims 4 to 6 to a subject in need thereof.

13-14. (canceled)

15. The method of claim 12, wherein the stem cells are human nasal inferior turbinate-derived stem cells (hNTSCs).

16. The method of claim 12, wherein the rheumatoid arthritis is collagen-induced arthritis.


SEQUENCE LISTING (48 sequences)

SEQ ID NO: 1 - DNA, 20 bp, Artificial Sequence
  SSTR1 F primer
tgagtcagctgtcggtcatc

SEQ ID NO: 2 - DNA, 20 bp, Artificial Sequence
  SSTR1 R primer
ggaaagagcgcttgaagttg

SEQ ID NO: 3 - DNA, 25 bp, Artificial Sequence
  SSTR1 probe
tatgccaacagctgcgccaacccca

SEQ ID NO: 4 - DNA, 22 bp, Artificial Sequence
  HAS2 F primer
ctgggacgaagtgtggattatg

SEQ ID NO: 5 - DNA, 20 bp, Artificial Sequence
  HAS2 R primer
gatgaggctgggtcaagcat

SEQ ID NO: 6 - DNA, 30 bp, Artificial Sequence
  HAS2 probe
aggtttgtgattcagacactatgcttgacc

SEQ ID NO: 7 - DNA, 20 bp, Artificial Sequence
  CXCL1 F primer
tcctgcatcccccatagtta

SEQ ID NO: 8 - DNA, 20 bp, Artificial Sequence
  CXCL1 R primer
cttcaggaacagccaccagt

SEQ ID NO: 9 - DNA, 25 bp, Artificial Sequence
  CXCL1 probe
cttcctcctcccttctggtcagttg

SEQ ID NO: 10 - DNA, 22 bp, Artificial Sequence
  CPED1 F primer
gtgaaacatctactctgggacc

SEQ ID NO: 11 - DNA, 22 bp, Artificial Sequence
  CPED1 R primer
ttaaatgctcgtgtacctggag

SEQ ID NO: 12 - DNA, 28 bp, Artificial Sequence
  CPED1 probe
ttggttatggcagtttcatgtaccctgt

SEQ ID NO: 13 - DNA, 19 bp, Artificial Sequence
  KRTAP1-5 F primer
tgagcccacttgctgaaag

SEQ ID NO: 14 - DNA, 20 bp, Artificial Sequence
  KRTAP1-5 R primer
tgtagcatttctgtgtcccc

SEQ ID NO: 15 - DNA, 25 bp, Artificial Sequence
  KRTAP1-5 probe
actgttcatcccttgaccacctctg

SEQ ID NO: 16 - DNA, 16 bp, Artificial Sequence
  GSTT2B F primer
gcagccggtggctctc

SEQ ID NO: 17 - DNA, 20 bp, Artificial Sequence
  GSTT2B R primer
aagatgatgctgtgggcctc

SEQ ID NO: 18 - DNA, 26 bp, Artificial Sequence
  GSTT2B probe
atgaactgtttgagggacggccacga

SEQ ID NO: 19 - DNA, 20 bp, Artificial Sequence
  OR10G2 F primer
tcctcagtcaccgttcctct

SEQ ID NO: 20 - DNA, 20 bp, Artificial Sequence
  OR10G2 R primer
aacctcccattcatgagcac

SEQ ID NO: 21 - DNA, 29 bp, Artificial Sequence
  OR10G2 probe
ccgccagttgcttcatgttaattctgctc

SEQ ID NO: 22 - DNA, 20 bp, Artificial Sequence
  DLCK1 F primer
gacatggagctggagcactt

SEQ ID NO: 23 - DNA, 20 bp, Artificial Sequence
  DLCK1 R primer
cttcttctcggagctgagcg

SEQ ID NO: 24 - DNA, 25 bp, Artificial Sequence
  DLCK1 probe
tcactggccactgccaaaggaagcc

SEQ ID NO: 25 - DNA, 20 bp, Artificial Sequence
  ADAMTS10 F primer
ctcatgttcgaggtcacgca

SEQ ID NO: 26 - DNA, 21 bp, Artificial Sequence
  ADAMTS10 R primer
cactttgtagaagaggcggga

SEQ ID NO: 27 - DNA, 26 bp, Artificial Sequence
  ADAMTS10 probe
atgagttcctgtccagtctggagagc

SEQ ID NO: 28 - DNA, 21 bp, Artificial Sequence
  SULF2 F primer
tgaaaggcaggtttcagaggg

SEQ ID NO: 29 - DNA, 20 bp, Artificial Sequence
  SULF2 R primer
gtggtcacgaaggcgttgat

SEQ ID NO: 30 - DNA, 26 bp, Artificial Sequence
  SULF2 probe
aggaacatccgccccaacatcatcct

SEQ ID NO: 31 - DNA, 18 bp, Artificial Sequence
  CFD F primer
gacaccatcgaccacgac

SEQ ID NO: 32 - DNA, 18 bp, Artificial Sequence
  CFD R primer
gttgactatgccccagcc

SEQ ID NO: 33 - DNA, 21 bp, Artificial Sequence
  CFD probe
agctgtcggagaaggccacac

SEQ ID NO: 34 - DNA, 25 bp, Artificial Sequence
  MGP F primer
cccttcattaacaggagaaatgcaa

SEQ ID NO: 35 - DNA, 19 bp, Artificial Sequence
  MGP R primer
gcgtagcgttcgcaaagtc

SEQ ID NO: 36 - DNA, 27 bp, Artificial Sequence
  MGP probe
tcccctcagcagagatggagagctaaa

SEQ ID NO: 37 - DNA, 20 bp, Artificial Sequence
  C4B F primer
gtggcctccatcaactcctc

SEQ ID NO: 38 - DNA, 20 bp, Artificial Sequence
  C4B R primer
tgtaaatgggctggtccgtc

SEQ ID NO: 39 - DNA, 23 bp, Artificial Sequence
  C4B probe
aggtccagctggtggcccattcg

SEQ ID NO: 40 - DNA, 20 bp, Artificial Sequence
  CCN5 F primer
cctcctctgcctcctctcaa

SEQ ID NO: 41 - DNA, 18 bp, Artificial Sequence
  CCN5 R primer
gacgtggagttggtcgca

SEQ ID NO: 42 - DNA, 24 bp, Artificial Sequence
  CCN5 probe
acccagctgtgcccgacaccatgt

SEQ ID NO: 43 - DNA, 20 bp, Artificial Sequence
  RBP1 F primer
ttgcgcaaaatcgccaactt

SEQ ID NO: 44 - DNA, 20 bp, Artificial Sequence
  RBP1 R primer
gcggtcatctatgcctgtca

SEQ ID NO: 45 - DNA, 26 bp, Artificial Sequence
  RBP1 probe
aaagagatcgtgcaggacggtgacca

SEQ ID NO: 46 - DNA, 20 bp, Artificial Sequence
  beta-actin F primer
ggacttcgagcaagagatgg

SEQ ID NO: 47 - DNA, 22 bp, Artificial Sequence
  beta-actin R primer
tgtgttggggtacaggtctttg

SEQ ID NO: 48 - DNA, 28 bp, Artificial Sequence
  beta-actin probe
taaggagaatggcccagtcctctcccaa